(19) United States
(12) Patent Application Publication
(10) Pub. No.: US 2005/0016538 A1
(43) Pub. Date: Jan. 27, 2005

(54) Control group of a second-stage regulator for scuba divers

(75) Inventor: Stefano Pedemonte, Ceranesi (IT)

Correspondence Address: Grant E. Pollack, Esq.;Steinberg & Raskin, P.C., 15th Floor, 1140 Avenue of the Americas, New York, NY, 10036-5803, US

(73) Assignee: Cressi-Sub S.p.A.

(21) Appl. No.: 10/899,203

(22) Filed: Jul. 26, 2004

(30) Foreign Application Priority Data
Jul. 25, 2003 (IT) FI2003U000070

(51) Int. Cl. A61M 16/00; A62B 7/04

(52) U.S. Cl. 128/204.26

(57) ABSTRACT

A group for the control of a second-stage regulator for scuba divers. The regulator comprises a regulator body with an inlet conduit for connection to a first-stage regulator which supplies a breathable gaseous mixture at a relatively constant pressure, and an outlet conduit for connection to a mouthpiece of the user. The group comprises a first bushing defining an intermediate chamber with a bottom wall wherein a central hole is formed, a poppet loosely engaged within the hole, the end of the poppet being connected to a lever, a second bushing engaged in the first bushing and defining an inlet chamber, and a third bushing engaged in the second bushing and defining a regulator seat at one of its ends. The head of the poppet engages tightly in the seat and an elastic member is provided coaxial to the poppet in the intermediate chamber for maintaining the head against said regulator seat, the group being removably engaged in the inlet conduit.

### FIELD OF THE INVENTION

The present invention relates generally to diving equipment and more particularly refers to a group for the control of a second-stage regulator for scuba divers.

### BACKGROUND ART

It is known that the supply of air, or of the air-oxygen mixture, which is fed to the mouthpiece of the scuba diver from a high-pressure tank, passes via a first stage pressure-reducing regulator to a second-stage regulator which supplies the mixture to the mouthpiece of the scuba diver when pressure within the regulator is diminished by a diver's inhalation.

Second-stage regulators of the known type have an inlet chamber connected to the outlet of the first-stage regulator, and an outlet chamber connected to the mouthpiece of the user and separated from the outside environment by an elastically deformable diaphragm which blocks an opening formed in the regulator body. The diaphragm is connected via a lever to a poppet which closes off the passage between the two chambers. The pressure inside the inlet chamber is maintained constant at approximately ten bars as the pressure in the tank varies thanks to appropriate calibration of the first-stage regulator. When the user does not breathe, his or her lungs, the mouthpiece, the outlet chamber and the outside environment are at the same pressure. When the user inhales, a vacuum is created inside the outlet chamber and the diaphragm bends towards the interior of said chamber, moving the poppet, which normally closes the passage between the inlet chamber and the outlet chamber, to an opening position.

The opening of the passage between the inlet chamber and outlet chamber creates an overpressure in the outlet chamber, so that the diaphragm returns into the rest position, moving the lever and returning the poppet into the starting position wherein the passage between the inlet chamber and the outlet chamber is closed once again.

In second-stage regulators of the known type the regulator seat, on which the seal of the head of the poppet rests, is housed inside the inlet conduit of the regulator which is integral to the body of the same regulator. This configuration complicates the regulator calibration operations required during assembly to compensate the unavaoidable dimensional deviations, within the design tolerances, of the various components from the optimum values. Moreover minor maintenance work on the regulator, which could be performed without problems even by the user, instead requires intervention by a specialised technician in that the device has to be recalibrated each time.

### OBJECTS AND SUMMARY OF THE INVENTION

The object of the present invention is to provide a group for the control of a second-stage regulator for scuba diver with a structure such as to simplify the operations of calibration during assembly and which enables simple maintenance work to be performed by the user without entailing the need to disassemble the control mechanism.

This object is achieved with the group for the control of a second-stage regulator for scuba diver according to the present invention whose feature consists in that it comprises a first bushing defining an intermediate chamber with a bottom wall wherein a central hole is formed wherein a poppet engages loosely, the end of the poppet being connected to lever means, and a second bushing engaged in the first bushing and defining a regulator seat at one of its ends, on which the head of the poppet engages tightly, elastic means being provided coaxial to the poppet in the intermediate chamber for maintaining the head against the regulator seat, the whole group being removably engaged in the inlet conduit.

### BRIEF DESCRIPTION OF THE DRAWINGS

The invention will now be illustrated in greater detail by the following description of one of its embodiments by way of a non-limiting example, with reference to the accompanying drawings, wherein:

FIG. 1 is a front perspective view of the second-stage regulator for scuba diver incorporating the control group according to the invention;

FIG. 2 is a partially exploded perspective view of the regulator of FIG. 1;

FIG. 3 is a longitudinal view of the control group;

FIG. 4 is a longitudinal cross section of the control group of FIG. 3 taken along lines IV-IV.

### DETAILED DESCRIPTION OF THE INVENTION

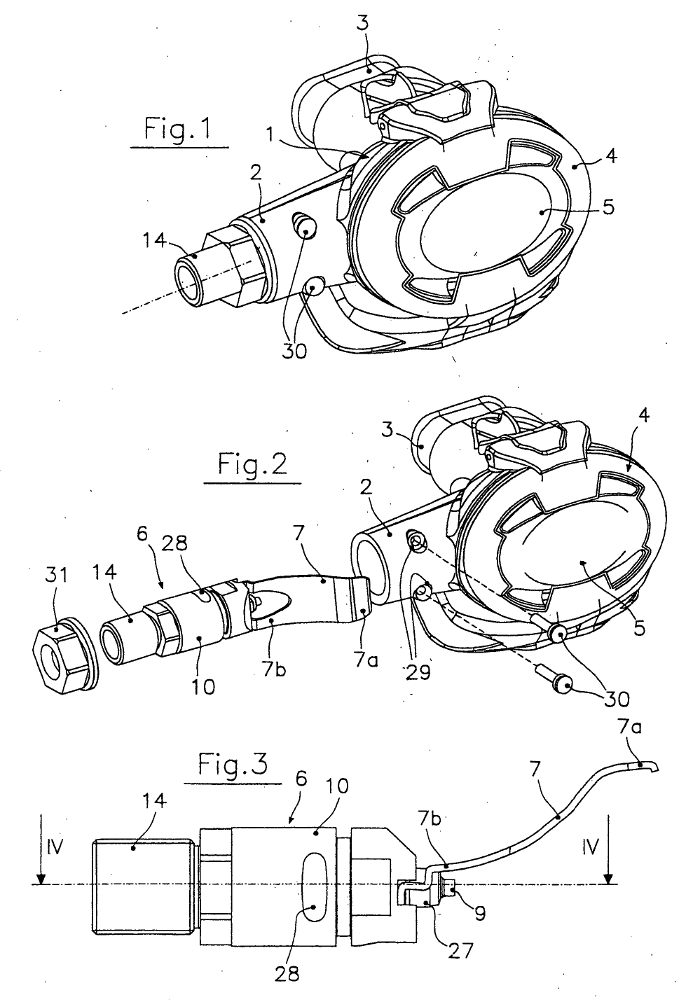

Referring to FIG. 1, 1 denotes the body of a second-stage regulator for scuba diver comprising an inlet conduit 2 and an outlet conduit 3. The inlet conduit 2 is used for connection to a first-stage regulator which supplies a breathable gaseous mixture at constant pressure, while the outlet conduit 3 is suitable for being held in the mouth by the scuba diver via a mouthpiece. The second-stage regulator comprises a front frame 4 attached to the regulator body 1 in any known manner so as to lock the internal diaphragm, not shown, against the edge of the corresponding opening, also not shown, of the regulator body 1, and holding a control pushbutton 5 centrally. The aforesaid parts are not described further in that they are well known to a person skilled in the art and in any case are not part of the specific area of the present invention.

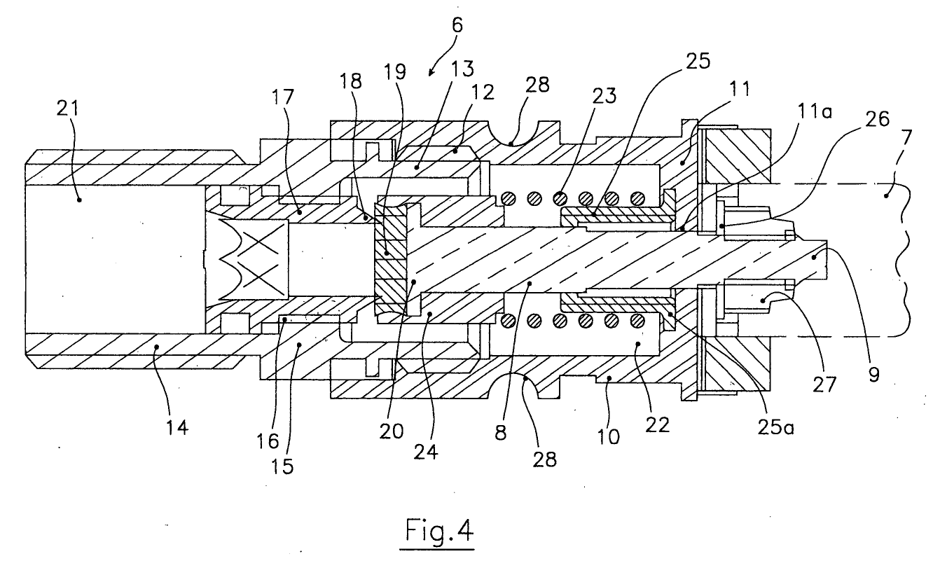

A regulator control group, shown in FIGS. 2, 3 and 4 and generically denoted by 6, is positioned inside the inlet conduit 2. The control group comprises a lever 7 which is in contact, in a known manner, with the aforesaid deformable diaphragm through its free end 7a, while with its other end 7b it is connected to the poppet of the regulator 8, and in particular to the end, or tail, 9 thereof, as shown in FIG. 3.

The control group 6 also comprises a first bushing 10 whose bottom 11 has a central hole 11a wherein the poppet 8 is engaged loosely. The first bushing 10 has internal threading 12 whereon an externally threaded end 13 of a second bushing 14 is screw engaged. Second bushing 14 in turn has an internal threaded portion 15 whereon a corresponding threaded portion 16 of a third bushing 17 is screw engaged. The end 18 of third bushing 17, turned towards the poppet 8, has an annular ribbing forming the regulator seat, on which a seal 19 of the head 20 of the poppet 8 is engaged.

The second bushing 14 defines an inlet chamber 21 wherein the third bushing 17 is engaged, while the first bushing 10 defines an intermediate chamber 22 communicating with the inlet chamber 21 through the regulator seat 18 cut off by the seal 19.

The seal 19 is kept forced against the regulator seat 18 by means of a coil spring 23 extending coaxially to the poppet 8 inside the intermediate chamber 22. In particular the spring 23 abuts on one side against a ring nut 24 which covers the head 20 of the poppet 8 and on the other side against a flange 25a of a tubular seal 25 connected tightly to the poppet 8 and to the bottom 11 of the first bushing 10 around the opening 11a.

The end 7b of the lever 7 is fork-shaped and engages on the tail 9 of the poppet 8 projecting from the hole 11a of the first bushing 10, between the bottom wall 11 and a washer 26. The washer 26 is tightened against the fork-shaped end 7b of the lever 7 by means of a nut 27 screwed to the tail 9 of the poppet 8.

The control group 6 is mounted as follows:
the tubular seal 25 is arranged at the bottom of the first bushing 10 and the spring 23 is rested on the flange 25a;
the ring nut 24 is inserted on the poppet 8 and then the tail 9 of the poppet is inserted first in the spring 23, then in the first bushing 10 and finally in the hole 11a of the bottom 11 thereof;
the washer 26 is inserted on the threaded end of the tail 9 of the poppet 8 and then the nut 27 is screwed on;
   the end 7b of the lever 7 is inserted between the washer 26 and bottom wall 11.

By adjusting the nut 27, the degree of tightness of the second bushing 14 inside the first bushing 10 and the degree of tightness of the third bushing 17 inside the second bushing, on the one hand it is possible to calibrate the force with which the seal 19 of the poppet 8 is pressed against the regulator seat 18 and, on the other hand, by adjusting the degree of tightness of the nut 27 it is possible to calibrate the exact position of the end 7a of the lever 7 intended to come into contact with the diaphragm, not shown, of the regulator.

The control group 6 described above can be calibrated by an appropriate equipment before mounting the group in the regulator body 1 via the inlet conduit 2 as shown in FIG. 2. Two diametrically opposite grooves 28 are formed on the external surface of the first bushing 10, perpendicular to the longitudinal axis of symmetry, and two holes 29 positioned at the same transverse distance as the grooves 28 are designed to hold two pins 30 when the aforesaid grooves are aligned with the holes 29. The relative longitudinal position of the group of elements of the second-stage regulator in relation to the inlet conduit 2 is therefore perfectly defined. The final attachment is achieved by means of a nut 31 which engages the external threading of the second bushing 14 until it abuts against the end of the inlet conduit 2.

Thanks to the structure of the control group for second-stage regulator according to the invention, the operation of calibration is simpler and more accurate in that it can be performed before mounting the group in the regulator body. It is also clear that by removing the entire group routine maintenance on the regulator is possible without having to dismount the group and therefore without having to calibrate it again, which would require assistance from a specialised technician.

Various modifications and alterations to the present invention may be appreciated based on a review of this disclosure. These changes and additions are intended to be within the scope and spirit of the invention as defined by the following claims.

1. A group for the control of a second-stage regulator for scuba divers, the regulator comprising a regulator body with an inlet conduit for connection to a first-stage regulator which supplies a breathable gaseous mixture at a relatively constant pressure, and an outlet conduit for connection to a mouthpiece of the user, wherein the control group comprises a first bushing defining an intermediate chamber with a bottom wall wherein a central hole is formed, a poppet loosely engaged within the hole, the end of the poppet being connected to a lever, a second bushing engaged in the first bushing and defining an inlet chamber, and a third bushing engaged in the second bushing and defining a regulator seat at one of its ends, the head of the poppet engaging tightly in the seat, an elastic member being provided coaxial to the poppet in the intermediate chamber for maintaining the head against the regulator seat, the group being removably engaged in the inlet conduit.

2. The control group set forth in claim 1, wherein the second bushing is threadably engaged in the first bushing and the third bushing is threadably engaged in the second bushing to allow regulation of their reciprocal position and, therefore, of the degree of compression of the elastic means.

3. The control group set forth in claim 1, further comprising an axial positioner between the first bushing and the regulator body.

4. The control group set forth in claim 3, wherein the axial positioner comprises at least one slot formed along the circumference of the first bushing and a pair of holes formed in the inlet conduit of the regulator body and aligned circumferentially, the holes being placed at a distance substantially equal to the length of the slot and being adapted for engagement with corresponding screws adapted for engagement with the slot so as to lock the position of the control group in relation to the regulator body.

5. The control group set forth in claim 4, wherein a plurality of diametrically opposite slots are formed in the bushing.

6. The control group set forth in claim 1, wherein the lever has a fork-shaped end engaged with the tail of the poppet projecting from the central hole formed in the bottom of the first bushing, the fork-shaped end being tightened against the bottom by a nut screwed to the tail of the poppet with the insertion of a locking washer.

7. The control group set forth in claim 1, wherein the elastic member comprises a coil spring extending between the bottom and the head of the poppet.

8. The control group set forth in claim 7, wherein a tubular seal is provided, connected tightly to the bottom and to
